(19) United States
(12) Patent Application Publication
(10) Pub. No.: US 2024/0342002 A1
(43) Pub. Date: Oct. 17, 2024

(54) PANNICULUS COMPRESSION UNDERGARMENT WITH NEOPRENE HEAT HOLDING COMPRESSION INSERTS

(71) Applicant: Josiah James Lenzi, Sarver, PA (US)

(72) Inventor: Josiah James Lenzi, Sarver, PA (US)

(21) Appl. No.: 18/299,903

(22) Filed: Apr. 13, 2023

(51) Int. Cl. A61F 7/02; A41B 9/00; A41B 9/14

(52) U.S. Cl. CPC A61F 7/02; A41B 9/001; A41B 9/14; A61F 2007/0233

(57) ABSTRACT

A panniculus compression undergarment includes a waistband; a pouch stitched to an interior surface of the waistband; and a heat retaining compression pad contained within the pouch. The waistband encircles a portion of a wearer's torso including an upper pubic area. The waistband, pouch, and heat retaining compression pad apply palpable pressure against the upper pubic area. The undergarment produces a permanent slimming effect of the panniculus area through targeted compression and heat holding materials.

### BACKGROUND OF THE INVENTION

The present invention relates to undergarments and, more particularly, to a Panniculus compression undergarment with neoprene heat holding compression inserts.

Panniculus or FUPA (Fat Upper Pubic Area), is a problem for both women and men and refers to a growth of dense, fatty tissue on the lower abdomen that sometimes hangs over the pubis and genitals. Both women and men can be predisposed to panniculus genetically. Women struggle with panniculus, especially after childbirth or weight-loss. Men with panniculus can lose up to one inch of visibility of their penis for every thirty pounds of weight that they gain, and panniculus can persist even after weight-loss. Other shapewear (primarily for women) only provides overall compression with materials like spandex for an immediate slimming effect. These products do not affect long-term slimming to the part of the body where panniculus occurs. No underwear on the market provides compression and heat holding features specific for the fat upper public area for men or women.

As can be seen, there is a need for an undergarment that slims the panniculus.

### SUMMARY OF THE INVENTION

In one aspect of the present invention, a panniculus compression undergarment comprises a waistband operative to encircle a portion of a wearer's torso including an upper pubic area; a pouch stitched to an interior surface of the waistband; and a heat retaining compression pad contained within the pouch. The waistband, pouch, and heat retaining compression pad cooperate to apply palpable pressure against the upper pubic area.

The FUPA Compressor produces a permanent slimming effect of the panniculus area through targeted compression and heat holding materials. Both women and men can use this product as an alternative to liposuction surgery or after surgery is completed to promote the flattening of the panniculus area. The FUPA compressor may be used alone or in conjunction with diet and exercise to increase the slimming of the targeted area. The FUPA Compressor can help restore the upper pubic area back to its previous form after childbirth.

Targeted compression may alternatively be used for the slimming or shaping of other portions of the human body.

These and other features, aspects and advantages of the present invention will become better understood with reference to the following drawings, description, and claims.

### BRIEF DESCRIPTION OF THE DRAWINGS

FIG. 1 is a perspective view of a panniculus compression device according to an embodiment of the present invention, shown in use;

FIG. 2 is a perspective view thereof;

FIG. 3 is an exploded view thereof;

FIG. 4 is a detail view thereof;

FIG. 5 is a rear perspective view thereof;

FIG. 6 is a sectional view thereof, taken along line 6-6 on FIG. 1, shown with the panniculus compressed;

FIG. 7 is another sectional view thereof, showing the panniculus decompressed; and

FIG. 8 is a perspective view of a panniculus compression device according to another embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a panniculus compression undergarment containing neoprene heat-holding compression inserts. The undergarment comprises a waistband, leg straps, and a pouch stitched to an interior surface of the waistband and operative to contain the compression insert. The undergarment applies palpable pressure against the upper pubic area.

The undergarment disclosed herein is sometimes referred to as the “FUPA Compressor”. This garment provides targeted compression to flatten the panniculus and holds heat at the panniculus area to promote the dissipation of fat cells and water to slim the panniculus.

The waistband encircles a portion of the wearer's torso and provides support to keep the compression undergarment in place on the body. The waistband is widened as compared to prior art undergarments to provide compression of the neoprene pad onto as much of the panniculus area as possible.

Leg straps pull the compression pad downwards and back into the body which results in compression to the lower part of the panniculus area. The leg straps also help keep the undergarment securely located on the panniculus region of the body. They generally extend from the front to both sides of the waistband but may extend from a front to a rear of the waistband.

The pouch is located on the interior front of the waistband to house the neoprene compression and heat holding pad. Its is strategically placed for maximum compression, while being visually unnoticeable.

In some embodiments, the pouch may be stitched permanently closed to the waistband, enclosing the heat retaining compression pad. In other embodiments, the pouch may have a Velcro@ hook and loop fastener or closure tab at the top to releasably fasten the compression pad within the pouch and to allow for thicker or thinner compression pads to be exchanged depending on preference.

The compression pad provides firm compression while staying pliable for comfort. It also holds heat at the targeted area to promote the dissipation of fat cells. The compression pads can be thinner or thicker depending on the user's selected intensity.

In some embodiments, the compression pad may have a semi-circular form at a bottom edge to expand the panniculus coverage area for men, as it also occurs around the sides of the male genitalia.

The compression pad is generally made of a 100% neoprene foam core for pliability and compression, and is encased in a 100% neoprene embossed waterproof fabric to entrap heat and secure the pad at the target area. It may have dimensions of, e.g., about 4 inches by about 4 inches, with varying thicknesses to vary intensity.

The waistband and leg straps may comprise an elastic fabric blend containing 70% polyester and 30% latex. The pouch fabric may be a blend such as 55% nylon, 25% latex and 20% cotton. The waistband may be, for example, about 4 inches wide to 8 inches wide depending on size. The components may be manufactured from a material selected from the group consisting of polyester, nylon, cotton, latex, rubber, and any combination thereof.

The leg straps are generally sewn to the front and sides of the waistband.

In some embodiments, leg straps may extend from front to back to allow the compression undergarment to be worn in women's panties, thongs, and boy-shorts. The pouch is sewn to the interior of the front center of the waistband, enclosing 100% of the neoprene compression and heat-retaining pad, for example, with stitching or Velcro® hook and loop closures.

The FUPA Compressor may be used to help slim a user's panniculus area by donning the undergarment, which may be worn any time day or night. Nighttime use may be preferred as the pad lays flatter on the targeted area, and with less movement, it stays more securely in place. The more the product is used, the better the results. For men, the undergarment may be worn inside boxers, briefs, and pouch underwear for discretion and to add comfort, support, and compression, with the look of traditional underwear. For women, the undergarment may be worn in women's panties, thongs, and boy-shorts.

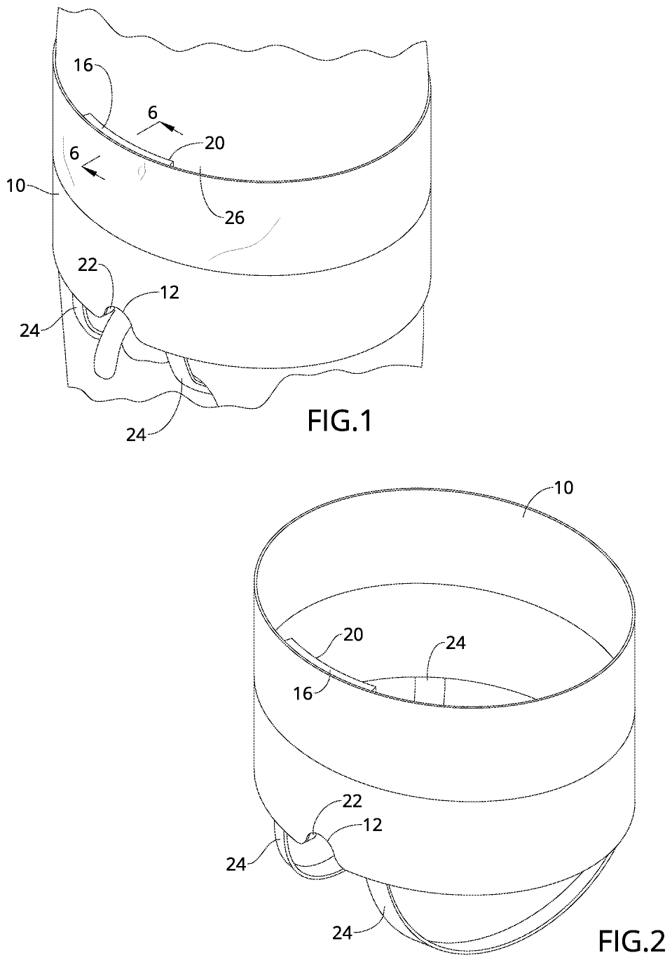

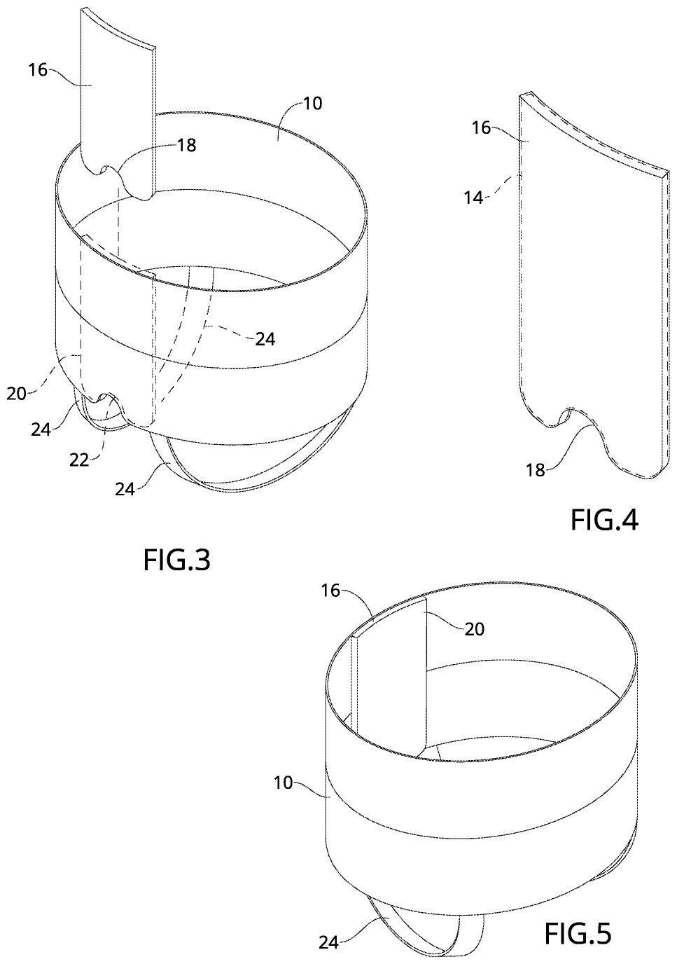

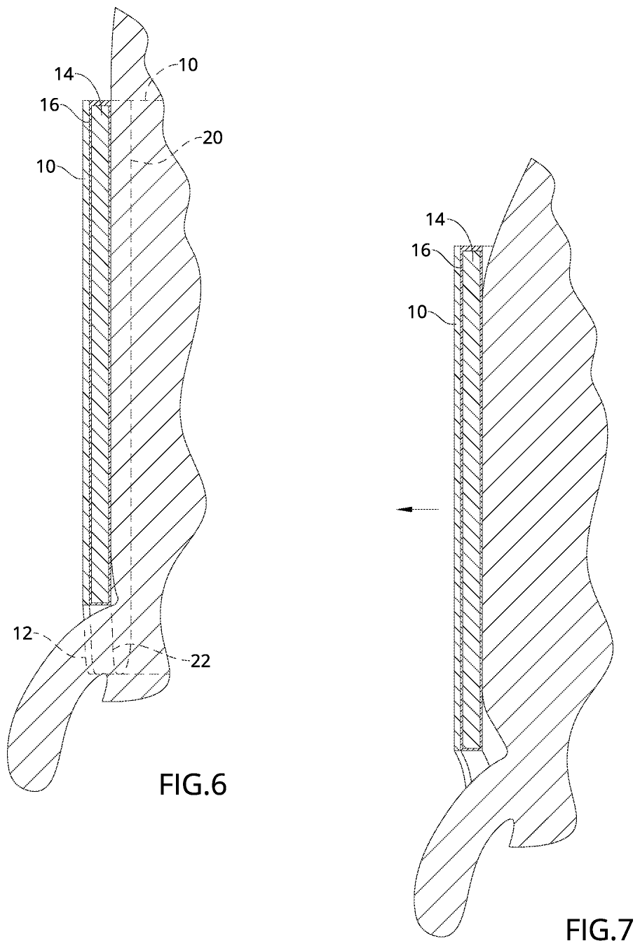

Referring to FIGS. 1 through 8, a compression undergarment according to an embodiment of the present invention is shown. As seen in FIG. 1, the garment comprises a waist band 10 with a pouch 20. A pad cover 16 configured to accommodate a compression pad 14 (see FIGS. 4 and 6-7) inserted into the pouch 20 (See FIGS. 3 and 5) is tightly held against the user's panniculus 26 by way of the waist band 10 and straps 24 affixed to the bottom of the waist band 10 on each side from front to rear, as shown in FIGS. 2 and 3. In this embodiment, a concave notch in the compression pad 14 at the groin and a corresponding waist band notch 12 located proximate to male genitalia enables compression at the groin. The pad cover 16 is formed with a pad cover notch 18 and the pouch 20 is formed with a pouch notch 22, respectively. Embodiments lacking the notches are also envisioned.

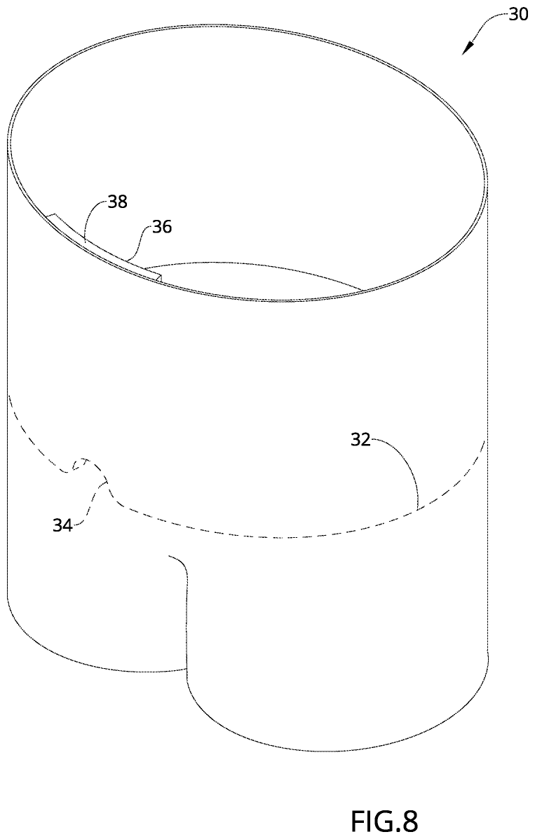

The undergarment may be contained within a second garment 30, such as long briefs, boxers, or shorts, according to another embodiment of the present invention as shown in FIG. 8. The garment includes a waist band 32 with a waist band notch 34 and pouch 36, with a covered pad 38 contained therein. Leg straps may be absent in this embodiment as the exterior undergarment functions to hold the undergarment in place.

It should be understood, of course, that the foregoing relates to exemplary embodiments of the invention and that modifications may be made without departing from the spirit and scope of the invention as set forth in the following claims.

What is claimed is:

1. A panniculus compression undergarment comprising:
a waistband operative to encircle a portion of a wearer's torso including an upper pubic area;
a pouch stitched to an interior surface of the waistband; and
a heat retaining compression pad contained within the pouch;
wherein the waistband, pouch, and heat retaining compression pad cooperate to apply palpable pressure against the upper pubic area.

2. The panniculus compression undergarment of claim 1, further comprising leg straps affixed to a bottom edge of the waistband extending from front to sides or from front to rear.

3. The panniculus compression undergarment of claim 1, further comprising a fastener releasably affixing a top of the pouch to a top of the waistband.

4. The panniculus compression undergarment of claim 1, wherein the waistband, the pouch, and the heat retaining compression pad are formed with a concave notch at a groin region thereof.

5. The panniculus compression undergarment of claim 1, wherein the heat retaining compression pad is about 4 inches wide by about 4 inches tall.

6. The panniculus compression undergarment of claim 1, wherein the heat retaining compression pad comprises neoprene.

7. The panniculus compression undergarment of claim 1, further comprising a pad cover configured to accommodate the heat retaining compression pad contained within the pouch.

8. The panniculus compression undergarment of claim 1, wherein further comprising a second garment housing the waistband, the pouch, and the heat retaining compression pad, the second garment selected from the group consisting of panties, thongs, boy-shorts, briefs, boxers, and shorts.

9. The panniculus compression undergarment of claim 1, wherein the waistband, the pouch, and the leg straps comprise an elastic fabric blend.

10. The panniculus compression undergarment of claim 9, wherein the elastic fabric blend comprises materials selected from the group consisting of polyester, nylon, cotton, latex, rubber, and any combination thereof.
